(19) United States
(12) Patent Application Publication
(10) Pub. No.: US 2020/0000503 A1
(43) Pub. Date: Jan. 2, 2020

(54) ACETABULAR COMPRESSION PLATE WITH DUAL CORTICAL FIXATION AND METHOD OF FIXING THEREOF

(71) Applicant: Ramesh Kumar SEN, West Chandigarh (IN)

(72) Inventor: Ramesh Kumar SEN, West Chandigarh (IN)

(21) Appl. No.: 16/341,588

(22) PCT Filed: Oct. 13, 2017
(86) PCT No.: PCT/IN2017/050469

§ 371 Date: Apr. 12, 2019

(30) Foreign Application Priority Data
Oct. 15, 2016 (IN) 201611035335

(51) Int. Cl. A61B 17/80; A61B 17/86

(52) U.S. Cl. CPC A61B 17/8066; A61B 17/8014; A61B 2017/564; A61B 17/864; A61B 17/8057

(57) ABSTRACT

A plate having unique anatomical brim specific shape and enables dual cortical fixation i.e. fixation from outer cortical region and inner cortical region, in a quick and easy manner is disclosed. Dual cortical fixation using nut and bolt enables compression (grip), enabling repair of fractures in normal as well as osteoporotic bone. The plate rests on to brim of pelvic so that a single screw or more screws can be inserted into it from the outer cortex of the bone that comes out of the brim, as brim is the strongest portion and holds the screws well. The screws are held into the bone using bi-cortical fixation by means of cannulated screws which are provided with washers to increase surface area of contact with bone. Owing to ease of use, the plate drastically reduces surgery time from few hours to less than an hour.

### FIELD OF INVENTION

The following invention relates to the field of medical devices, especially those which are used in orthopedic surgery. More specifically the present invention relates to a novel acetabular compression plate with dual cortical fixation and method of fixing thereof. The plate is suitable for reconstructing acetabular fractures especially in osteoporotic bone and is made up of suitable metal such as medical grade stainless steel, titanium or the like.

### BACKGROUND OF THE INVENTION

Fractures of the hip region (acetabulum) are common due to accidents, falls etc. and are very difficult to repair. Existing methods involve use of metal ‘plates’ which need to be fixed with screws in the hip region.

Limitations of existing plates
i. Loosening with passage of time—Plates become loose after some time as fixing is from one side only. With passage of time the fixing screws lose their grip due to which the plates become loose. Existing plates have no provision for adequate bi-cortical fixation/purchase. They can be fixed from inner cortical side only and not from outer cortical side due to which grip is less than if the plates are fixed from both sides i.e. inner as well as outer cortex.
ii. Not suitable for fracture repair in osteoporotic bone—Repair of fracture in case of bone which has been weakened due to calcium loss (osteoporotic bone) is very difficult, since screws are difficult to fix in the weakened bone. Existing plates are fixed or held in place from one side only i.e. passing of screws on the inner cortex (inner cortex refers to surface of bone facing towards inner surface of body). In case the bone is osteoporotic, then the single cortex fixation method is not suitable because the screws do not maintain their grip with bone leading to loosening of the plate with passage of time.
iii. Fixation is very cumbersome and time consuming—Fixation of plates in the hip region (acetabulum) in case of fracture, is a time consuming, cumbersome and difficult process for the surgeon, leading to long surgery time. This is because none of the existing plates are designed for easy fit in pelvic region.

Limitations Addressed by Plate of Present Invention
i. Does not become loose-Plate does not become loose since it is fixed from two sides (dual cortical fixation)
ii. Can be used in case of osteoporotic bone also—Plate can be used to repair hip region fractures in case of osteoporotic bone also since it is anatomical on inside and grip (compression) is very good due to fixation from two sides (dual cortical fixation)
iii. Fixation is easy and quick—Fixation of the plate is quick, easy and efficient, leading to drastically reduced surgery time (from 2.5-3 hours in case of traditional methods to less than 1 hour in present method). This is due to anatomical shape of the plate, corresponding to the pelvic brim.

To promote understanding of the invention, technical terms used in context of present invention are described below:
i. Pelvis: This is either the lower part of the trunk of the human body between the abdomen and the thighs (sometimes also called pelvic region of the trunk) or the skeleton embedded in it (sometimes also called bony pelvis, or pelvic skeleton). The pelvic region of the trunk includes:
   the bony pelvis
   the pelvic cavity (the space enclosed by the bony pelvis)
   the pelvic floor (below the pelvic cavity)
   the perineum (below the pelvic floor).
ii. Pelvic skeleton is formed in the area of the back, by the sacrum and the coccyx and anteriorly and to the left and right sides, by a pair of hip bones. The two hip bones connect the spine with the lower limbs. They are attached to the sacrum posteriorly, connected to each other anteriorly, and joined with the two femurs at the hip joints. https://en.wikipedia.org/wiki/Pelvis
iii. Acetabulum (hip socket) is a concave surface of the pelvis. It is the cup-shaped cavity on the lateral surface of the hip bone and formed by the coming together of three bones of the hip bone viz. ilium—the broad, flaring upper portion; ischium—the thick three sided part behind and below the acetabulum and pubis consisting of body (anterior expanded portion), inferior ramus and superior ramus. The boundary of aperture leading into true pelvis is called the pelvic brim. Reference: https://en.wikipedia.orgwiki/Acetabulum
iv. Iliopectineal eminence or Iliopubic eminence: It is anterior to acetabulum and is another strong area around acetabulum.
v. Acetabular fracture is a break in the socket portion of the “ball-and-socket” hip joint. These hip socket fractures are not common, they occur much less frequently than fractures of the upper femur or femoral head (the “ball” portion of the joint). The acetabular fractures are generally caused by some type of high-energy event, such as a car collision. In some cases acetabular fractures are sometimes caused by weak or insufficient bone. This is most common in older patients whose bones have become weakened by osteoporosis. Treatment for acetabular fractures often involves surgery to restore the normal anatomy of the hip and stabilize the hip joint.
Reference: http://orthoinfo.aaos.org/topic.cfm?topic=A00511
vi. Osteoporosis is a bone disease that occurs when the body loses too much bone, makes too little bone, or both. As a result, bones become weak and may break from a fall or, in serious cases, from sneezing or minor bumps. Osteoporosis means “porous bone.” Viewed under a microscope, healthy bone looks like a honeycomb. When osteoporosis occurs, the holes and spaces in the honeycomb are much larger than in healthy bone. Osteoporotic bones have lost density or mass and contain abnormal tissue structure. As bones become less dense, they weaken and are more likely to break. Breaking of bone is a serious complication of osteoporosis, especially with older patients. Osteoporotic bone breaks are most likely to occur in the hip, spine or wrist, but other bones can break too.
Ref: https://www.nof.org/patients/what-is-osteoporosis/
vii. Kirachner wires or K-wiresor pins are sterilized, sharpened, smooth stainless steel pins. Introduced in 1909 by Martin Kirschner, the wires are now widely used in orthopedics and other types of medical and veterinary surgery. They come in different sizes and are used to hold bone fragments together (pin fixation) or to provide an anchor for skeletal traction. The pins can also be driven into the bone through the skin (percutaneous pin fixation) using a power or hand drill.
Ref: https://en.wikipedia.org/wiki/Kirschner_wire
viii. Cortical fixation/purchase refers to method of fixing of plates to the bone in which screws are inserted through the outer surface or ‘cortex’ of the bone. Cortical fixation/purchase is of two types:
   a. Single cortical fixation—Screws are inserted only from one side of the bone, from inner cortex and remain in the bone only and do not come out of other side of the bone. Hence, grip of the screw depends upon the strength of the bone only, since screw is embedded in the bone.
   b. Dual cortical fixation—The screws (bolt) are fixed from both sides, resulting in a very powerful grip and excellent ‘holding’ of the fractured parts. Dual fixation involves insertion of cannulated or “Hollow Screws” (bolt) from one side of the bone (in this case from outer or inner cortex) to come out of other side (in this plate through inner cortex or outer cortex and the across plate) and then this screw (bolt) is transfixed by another screw (nut) from other side which fits into the cavity of the first screw (bolt). This dual fixation method for acetabular fixation has been used by inventor for first time for repair of acetabular fracture. Using a jig through one of the holes in the pelvic bone (compression hole) a guide wire is drilled from the outer cortex or a guide wire is inserted from the inner cortex to come out of the outer cortex. Over the guide wire the cannulated screw (bolt) is inserted from the outer cortex. Then through the compression hole on the plate another (smaller) screw (nut) is inserted from inner cortex over the guide wire towards the outer screw (bolt). The inner screw (nut)/smaller screw drives on the internal thread in the cavity of the outer screw (bolt). This helps in increasing fracture reduction by compression across plate and outer cortex. This innovative approach enables proper grip to plate even in case of osteoporotic bone—a technical challenge which was not being addressed by prior art plates and their methods of fixation.

### INTRODUCTION TO THE PRESENT INVENTION

The present invention relates to a novel acetabular compression plate with dual cortical fixation and method of firing thereof for reconstructing acetabular fractures even in osteoporotic bone. The overall shape and parts of the plate are duly illustrated in FIG. 7 and consist of
upper extended portion (A)
two lower portions (B and C)
a central portion which rests over the brim (D) and is anatomically contoured to correspond to shape of pelvic brim
a notched part (N) which fits into the pelvic brim in the posterior part of the brim so that the plate fits anatomically over the brim and does not slide back, thus retaining its correct anatomical position.

The plate has a plurality of holes as illustrated in FIG. 8 comprising:
a plate holding screw hole (SH1)
a brim screw hole (SH2)
an anterior screw hole (SH3)
screw holes for additional fixation, if required (SH4).

Embodiments of the plate are represented by FIG. 11 and FIG. 12. The basic structure in the embodiments remains the same viz. upper part, lower parts and central brim part. However, there is variation in the holes (threaded holes vs simple holes without threads) and presence of additional small holes (for ball spikes, to keep plate in position) in embodiments. The holes are critically positioned to enable proper anatomical bi-cortical fixation of the plate.

Special Features of the Plate of Present Invention

The plate is contoured as per natural anatomical curve and shape of the brim of pelvic bone so that it can be easily placed on the pelvic brim and thus enable proper positioning for firm grip. Hence, it is called “anatomical plate”. When fixed to the bone, the novel plate provides three times strength to the hone compared to actual bone strength, making it an excellent product for repair of acetabular fractures. The anatomical features of the plate include:
a notched part and
a central portion which is anatomically contoured to correspond to shape of pelvic brim.

Notched part is to fit into pelvic brim in the posterior part of the brim, so that the plate fits anatomically over the brim and does not slide back, thus retaining its correct anatomical position. The central portion of the body is anatomically contoured to pelvic brim to enable correct positioning in quick and easy manner. A Screw line has been specifically created over the ridge of the pelvic brim to facilitate the screw insertion from inside towards the outer cortex. This brim screw line has one big hole for transfixation screw (dual cortical fixation nut bolt) and 3 small holes for 3.5 mm screws. Pubic arm will have 4-5 holes, as per plate size. Upper flange of the body is having multiple screw hole line where the first most posterior screw is to stabilize the plate for correct/optimum anatomical positioning of the plate. The body of the plate has two downward flanges (B) and (C)—1 for each acetabular column (FIG. 7) and plurality of holes. In a preferred embodiment (FIG. 14), the posterior downward flange is bigger and has three holes for screw insertion. The anterior one is small and has 2 holes only.

Technical Challenges Addressed by Plate of Present Invention:
1. Loosening of plates in case of osteoporotic bone: The plate of present invention does not become loose, since it is fixed from both sides i.e. inner and outer sides (dual cortical fixation). The novel and anatomically specific shape of the plate holds the fractured bone well in case of both osteoporotic bone and normal healthy bone.
2. Lack of proper grip: The plate of present invention offers unmatched strong grip due to dual cortical fixation/purchase method instead of single cortical fixation. For holding the plate properly with the bone and increasing its grip, bolt with nut and washers are used on the screw that increases the impact area of the screws on the bone surface. Also, the inventor has disclosed the use of fixing screws from the outer cortex of the bone in the pelvic region for the first time. Dual cortical fixation on the brim of the bone (purchase through both inner and outer cortex) offers technical advantage of better fixation because outer cortex is the strongest portion of the bone.
3. Difficulty of proper fixation in pelvic region: The novel plate is anatomical brim specific plate which provides stability and fixation to both upper and medial surface of the pelvic brim. The superior extended portion of the plate is so shaped that it can hold the multiple fractures of the acetabular region using single plate only.
4. Cumbersome fitting and prolonged surgery time: The plate of present invention and its unique, simplified mechanism of fitting drastically reduces surgery time from about 2.5-3 hours to less than one hour.

### PRIOR ART

| S. | Patent | | |
|---|---|---|---|
| No | application no. | Prior Art Invention | Present Invention |
| 1. | CN201831946U | FIG. 1 | FIG. 7 |
| | | The anatomical steel plate | The anatomical steel plate of |
| | | comprises a bent shape able plate | present invention comprises a |
| | | body provided with a plurality of | anatomical brim specific shape |
| | | locking holes which can be fixed | which enables dual cortical |
| | | from inner cortical only. | fixation i.e. fixation through |
| | | | outer cortical region and inner |
| | | | cortical region |
| 2. | 845/DEL/2009 | FIG. 2 | FIG. 7 |
| | | The present invention discloses a | The present invention discloses |
| | | fixation device/plate for fixation | a novel acetabular compression |
| | | and reconstruction of the | plate that is used to hold |
| | | fractures of the pelvic bone which | fractured bone properly even in |
| | | is applicable to most fractures of | the case of osteoporotic bone |
| | | the ilium where anterior column | which is not possible in plate of |
| | | is involved. The device has a | prior art. The plate of present |
| | | unique biological design, | invention is anatomical and |
| | | comprising of a single median | brim specific and provides |
| | | portion, with two lateral arms | better fixation strength to the |
| | | which fit snugly into the two | bone. Moreover the plate is |
| | | natural grooves on the inner | fixed using bi-cortical screws |
| | | surface of the iliac region of the | inserted from the outer cortex |
| | | pelvis. | of the bone and tightened |
| | | | from the inner cortex side or |
| | | | vice versa also. In contrast, the |
| | | | plate in prior art patent |
| | | | application is fixed using single |
| | | | cortex purchase that to from |
| | | | inner cortex of the bone as |
| | | | normally done. |
| 3. | EP2494934A1 | FIG. 3 | FIG. 7 |
| | | The invention relates to a pelvic | In the present invention bi- |
| | | bone plate implant having a | cortical purchase is used for |
| | | planar or curved outer frame | fixing the screws, but in the |
| | | portion. The plate has a flap | disclosed patent application |
| | | portion, the outer frame portion at | screws are implanted using |
| | | least partially surrounds the flap | single cortex fixation only. |
| | | portion such that the bone | In the present invention the |
| | | contacting surface the flap | inventor has designed a plate |
| | | portion is located within the outer | that is anatomic and brim |
| | | boundary of the frame portion. | specific with extended portion |
| | | | that hold the other multiple |
| | | | fractures near to the brim |
| | | | whereas the plate in disclosed |
| | | | prior art patent application is |
| | | | not pelvic brim specific i.e. it is |
| | | | not having anatomical shape. |
| 4. | US 20120010617 | FIG. 4 | FIG. 7 |
| | A1 | Systems for repairing acetabulum | In the present invention design |
| | | fractures can include a bone plate | of the plate is brim specific |
| | | that has openings therein and one | and multiple screw holes are |
| | | or more bolts sized to fit through | provided on the extended |
| | | the openings. The bone plate can | portion of the plate so as to |
| | | cooperate with the head portions | provided firm grip in case of |
| | | of the bolts to prevent them from | multiple fractures in acetabular |
| | | spinning within the holes as | region even for osteoporotic |
| | | screws are attached to the bolts. | patients. However, prior art |
| | | | plate does not contain any |
| | | | extended portions and is not |
| | | | suitable for osteoporotic bone. |
| 5. | US 20050165401 | FIG. 5 | FIG. 7 |
| | A1 | The plate primarily includes: an | The present invention is an |
| | | attachment member having at | anatomically designed brim |
| | | least one aperture formed therein: | specific plate that rests on the |
| | | an optional additional attachment | brim of the pelvic and multiple |
| | | member extending from the | screw holes are provided on the |
| | | attachment member, the | extended portion of the plate so |
| | | additional attachment member | as to provide firm grip to hold |
| | | having at least one aperture | multiple fractures in acetabular |
| | | formed therein; and a support | region. However in prior art |
| | | member extending angularly | patent application only a |
| | | from the attachment member. | support member is provided but |
| | | (FIG. 5) | it does not contain any holes |
| | | | through which screws can be |
| | | | inserted. Also, in prior art |
| | | | screws are implanted using |
| | | | single cortex fixation only |
| | | | whereas in present invention |
| | | | bi-cortical purchase is used |
| | | | for fixing the screws. |

LITERATURE REFERENCE

Guo-Chun Zhu et al. (2015) discloses a Novel Fixation System for Acetabular Quadrilateral Plate Fracture consisting of a segmental plate of inner wall. FIG. 6.

The plate is designed in terms of the shape and dimensions of quadrilateral plate, which is expanded to hold the quadrilateral plate. The proximal portion of the plate has an extended arm with a ratchet, a segmental plate of ilium. The distal portion of the plate has a space which connects the ratchet; a connecting arm that links the ratchet to the segmental plate of ilium, which forms an articulated joint; a set bolt: a pawl that can cooperate with the ratchet and the set bolt, forming unidirectional articulated joint from medial to lateral. (Guo-Chun Zha, Jun-Ying Sun, Sheng-Jie Dong. Wen Zhang, and Zong-Ping Luo, “A Novel Fixation System for Acetabular Quadrilateral Plate Fracture: A Comparative Biomechanical Study,” BioMed Research International, vol. 2015, Article ID 391032, 8 pages, 2015. doi:10.1155/2015/391032).

In contrast, the plate of present invention (FIG. 7) is quite different and is a single piece anatomically designed brim specific plate. The plate is fixed using bi-cortical fixation and that too from the outer cortex of the bone. Further the plate has anatomical shape which corresponds to shape of pelvic brim.

From the above it is clear that none of the prior art discloses or anticipates the plate of the present invention which is novel and innovative, has anatomical brim specific shape and offers easy, bi-cortical fixation.

### OBJECTS OF THE INVENTION

The main object of the invention is to disclose a novel anatomical acetabular compression plate with dual cortical fixation for reconstructing acetabular fractures in both osteoporotic bone and normal healthy bone.

Another object is to disclose an anatomical and brim specific novel acetabular compression plate with dual cortical fixation.

Still another object is to provide a novel acetabular compression plate with dual cortical fixation that maintains proper grip even with osteoporotic bone due to which it does not become loose and hence is not required to be replaced.

A further object is to disclose a method of fixing of novel acetabular compression plate using bi-cortical fixation in pelvic region.

Yet another object is to disclose a novel acetabular compression plate that allows dual cortical fixing using cannulated screws such that one screw is inserted from the outer cortex of the bone in the pelvic region and the second screw is inserted from the inner cortex and fits into the hollow cavity of the other screw for maintaining proper grip and compression.

A further object is to disclose a novel acetabular compression plate which can be fixed easily, quickly and accurately thus drastically reducing surgery time from a few hours to less than an hour.

A last object is to disclose a novel acetabular compression plate which on account of its unique features allows quick and easy fixation without need for too much training or skill development needed to train surgeons for this type of surgery.

### SUMMARY OF THE INVENTION

The present invention discloses a novel plate for repair of hip region (acetabular) fractures, which has unique anatomical brim specific shape (FIG. 7) and enables dual conical fixation i.e. fixation from outer conical region and inner cortical region, in a quick and easy manner, reducing surgery time. Dual cortical fixation (fixing from two sides of cortex i.e. inner side and outer side using nut and bolt) enables strong grip (compression), enabling repair of fractures in normal as well as osteoporotic bone in a very effective manner. The plate has extended portions on the superior and medial side of the brim which are anatomically shaped. Multiple fractures around the brim of acetabular region can be repaired by using single plate only. The plate is made to rest on to brim of pelvic so that a single screw or more screws can be inserted into it from the outer cortex of the bone that comes out of the brim, as brim is the strongest portion and holds the screws well. This offers good grip irrespective of whether the bone is normal or osteoporotic. The screws are held into the bone using bi-cortical fixation to maintain proper grip/compression. The cannulated screws are provided with washers to increase surface area of contact with bone. This offers the technical advantage that the screw nuts are prevented from being ‘buried’ inside the bone in due course of time due to pressure/movement of patient etc. and their proper position is maintained.

The plate is used where available standard plates do not provide satisfactory results e.g. in case of osteoporotic bone. Present invention is thus a low cost, reliable and efficient tool for orthopedic bone fracture repair surgery for acetabular region. Further, its fitting is easy and does not involve too much training, thus enabling quick and accurate fixation and also reducing surgery time drastically—from 2.5-3 hours to less than one hour.

### DESCRIPTION OF DRAWINGS

FIG. 1: Schematic diagram of already existing/prior art plate as disclosed in patent application number CN201831946U

FIG. 2: Schematic diagram of already existing/prior art plate as disclosed in patent application number 845/DEL/2009

FIG. 3: Schematic diagram of already existing/prior art plate as disclosed in patent application number EP249494A1

FIG. 4: Schematic diagram of already existing/prior art plate as disclosed in patent application number US 20120010617A1

FIG. 5: Schematic diagram of already existing/prior art plate as disclosed in patent application number US 20050165401A1

FIG. 6: Schematic diagram of already existing/prior art plate as disclosed by Gua-Chen et al (2015)

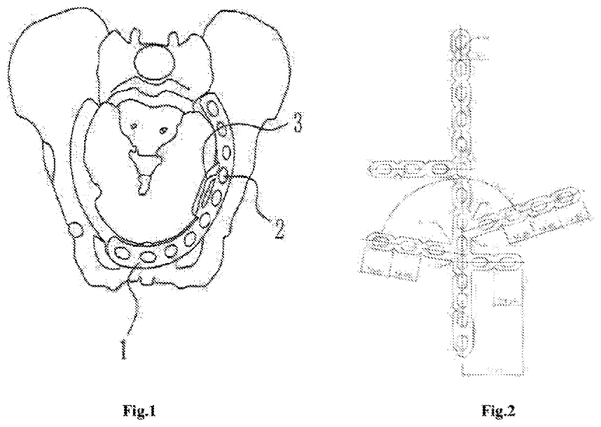

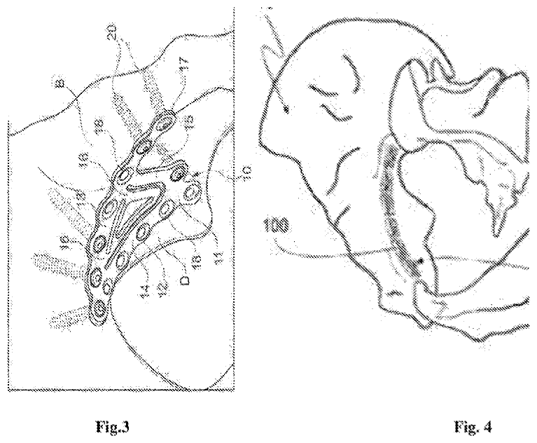

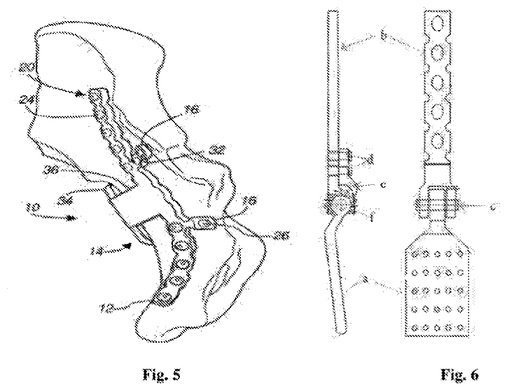

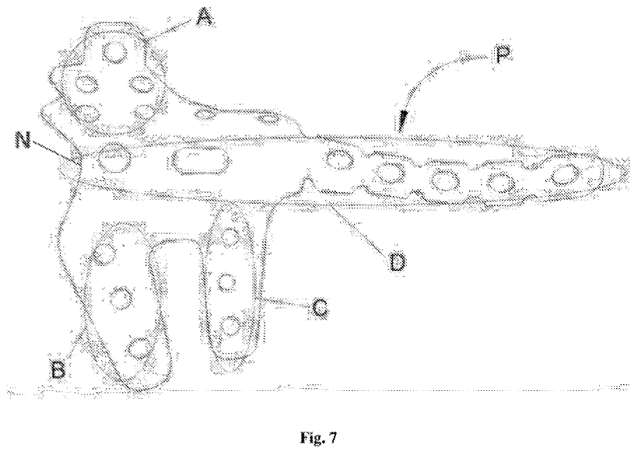

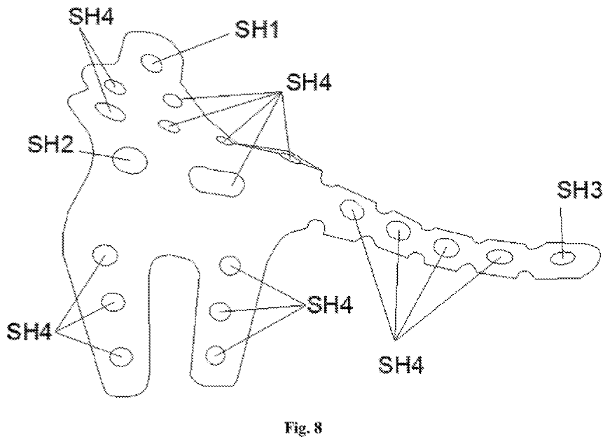

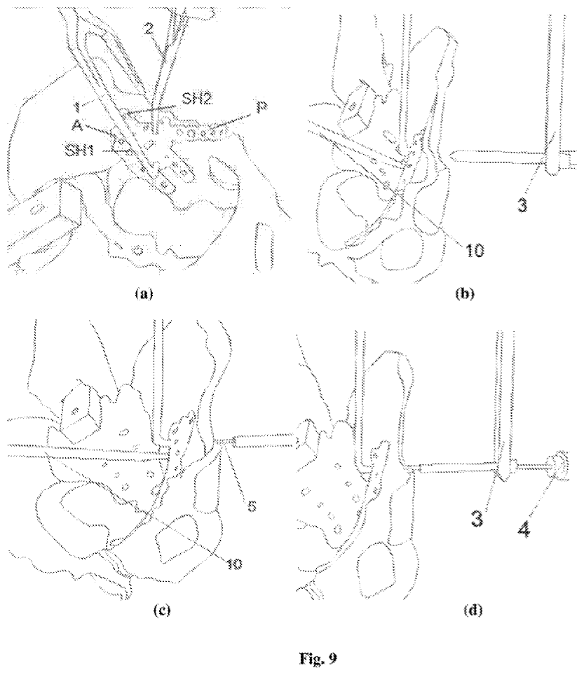

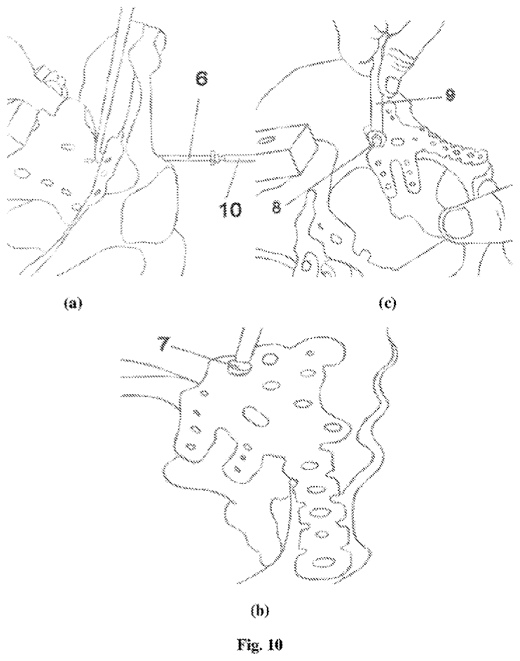

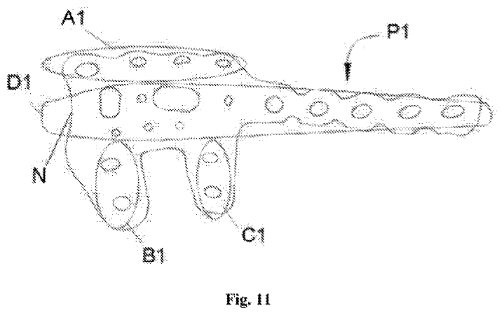

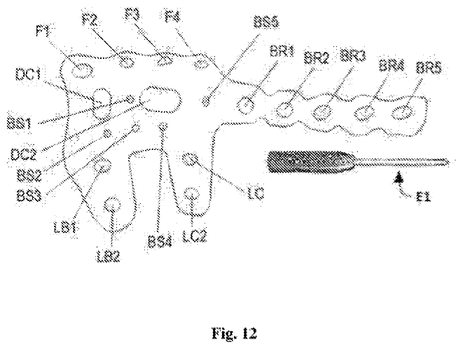

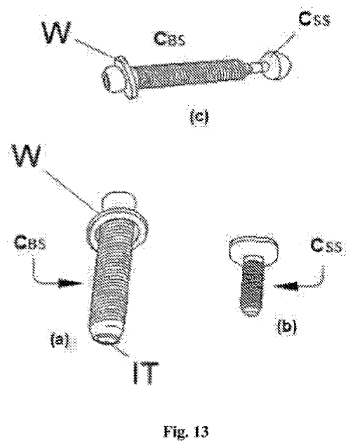

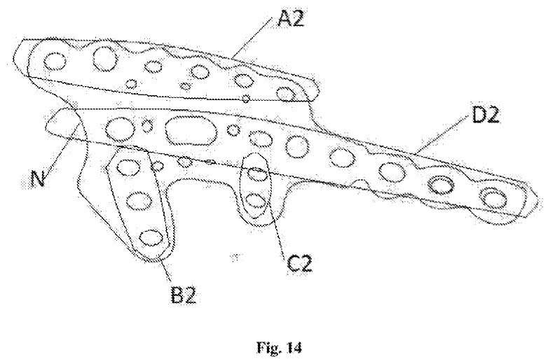

FIG. 7: Schematic diagram of acetabular plate (P) as used in the first embodiment of the present invention
A. Upper extended portion
B. Lower extended portion
C. Lower extended portion
D. Central Brim portion
N. Notched Part

FIG. 8: Schematic diagram of acetabular plate (P) showing types of holes in the plate
SH1—Plate Holding Screw Hole
SH2—Brim Screw Hole
SH3—Anterior Screw Hole
SH4—Screw holes for additional fixation if required.

FIG. 9a, b, c, d: Schematic diagram showing tools and accessories used for fixation of acetabular plate
1. Asymmetrical Clamp
2. Clamp forceps
3. Jig with compression sleeve and trocar
4. Drill with Drill Bit
5. Guide wire
6. Bi-cortical cannulated nut and bolt
7. Single cannulated cortical screw
8. Nuts
9. Spanner
10. Screw driver

FIG. 10: Schematic diagram showing acetabular plate after fixation

FIG. 11: Schematic diagram of second embodiment of acetabular plate and ball spike
A1. Flange
B1. Lower extended portion
C1. Lower extended portion
D1. Brim portion
E1. Ball Spike used for fixation
N. Notched Part

FIG. 12: Schematic diagram of acetabular plate of the second embodiment
F1—Locking (threaded) Screw hole
F2—Screw hole
F3—Screw hole
F4—Locking (threaded) Screw hole
BR1—Screw hole
BR2—Screw hole
BR3—Screw hole
BR4—Screw hole
BR5—Screw hole
BS1—Small Hole
BS2—Small Hole
BS3—Small Hole
BS4—Small Hole
BS5—Small Hole
LB1—Screw Hole
LB2—Screw Hole
LC1—Screw Hole
LC2—Screw Hole

FIG. 13: Schematic diagram of cannulated screw used in acetabular plate fixation
CS—Cannulated bolt fitted with smaller sized nut
C_{BS}—Cannulated Bigger (bolt) Screw
C_{SS}. Cannulated Smaller (nut) Screw
W—Washer

FIG. 14: Schematic diagram of acetabular plate of third embodiment
A2. Flange
B2. Lower extended portion
C2. Lower extended portion
D2. Brim portion
N. Notched Part

### DETAILED DESCRIPTION OF THE INVENTION

The present invention relates to a novel acetabular compression plate with dual cortical fixation and method of fixing thereof for reconstructing acetabular fractures even in osteoporotic bone.

The plate is contoured as per natural anatomical curve and shape of the brim of pelvic bone, so that it can be easily placed on the pelvic brim and thus enable proper positioning to give firm grip. Hence, it is called “anatomical plate”. When fixed to the bone, the novel plate provides three times strength to the bone compared to actual bone strength, making it an excellent product for repair of acetabular fractures.

The anatomical features of the plate include a notched part and a central portion which are anatomically contoured to correspond to shape of pelvic brim. Notched part (Part N of FIG. 7) is to fit into pelvic brim in the posterior part of the brim, so that the plate fits anatomically over the brim and does not slide back, thus retaining its correct anatomical position. The central portion of the body (Part D of FIG. 7) is anatomically contoured as per shape of pelvic brim to enable quick, easy and correct positioning.

A plurality of holes (screw line) has been specifically created in the central portion of the plate (P) of FIG. 7, which rests over the ridge of the pelvic brim to facilitate the screw insertion from inside towards the outer cortex. This brim screw line has single or plurality of big holes for insertion of transfixation screws (nut and bolt) and also plurality of small holes, preferably three, for 3.5 mm screws. The extended arm of brim portion (pubic arm) has plurality of holes, preferably 4-5, as per size of the plate.

Upper flange of the body is having plurality of screw holes (screw hole line). In an embodiment, the first and last screw holes (F1 and F4 of FIG. 12) are having locking mechanism (screw threads) to stabilize the plate for correct/optimum anatomical positioning of the plate.

The boxy has two downward flanges—1 for each acetabular column. The posterior one is bigger and has plurality of holes, preferably three for screw insertion. The anterior one is small and also has plurality of holes, preferably two for screw insertion.

In a first embodiment of the invention (FIG. 7 and FIG. 8), the acetabular plate (P) is fragmented into four portions viz.
i. Upper extended portion (A)
ii. Lower extended portion (B)
iii. Lower extended portion (C)
iv. Central Brim portion (D)

The central portion (D) of the acetabular plate (P) rests on the brim of the acetabular region in pelvic. This portion (D) is anatomically shaped and brim specific. The extended portions (A, B and C) of the plate have plurality of holes as illustrated in FIG. 8.

The upper extended portion (A) contains a plate holding screw hole (SH1) that initially supports and holds the plate. The lower extended portions (B and C) also contain holes (SH4) for inserting screws. The upper extended and lower extended portions are shaped as per the anatomy of the pelvic bone on which the plate rests. An anterior screw hole (SH3) is provided on the brim portion (D) of the plate to align the plate as per anatomical shape of the pelvic bone.

On the brim portion a brim screw hole (SH2) is provided into which bi-cortical screw (nut and bolt) is inserted from the inner or outer cortex of the bone. The bi-cortical screw contains a head with bolt on one side and is inserted into the bone (from outer cortex of bone) and other nut is inserted into the screw from the other end and is tightened to provide compression to the plate with the bone.

Tools used for fixing the plate as disclosed in first embodiment of the present invention, are depicted in FIG. 9.
1. Asymmetrical clamp (1) is used for fracture reduction. (FIG. 9 a)
2. The acetabular plate (P) is held with clamp forceps (2) and asymmetrical clamp (1) (FIG. 9 a).
3. Jig (3) for precise drilling into the bone cortex using drill with drill bits (4) (FIG. 9 d)
4. Guide wire (S) (FIG. 9 c)
5. Bi-cortical (bolt) or single cortical screws (6 and 7) (FIG. 10 a, b) with washers and nuts (8) FIG. 10 c, for holding the plate.
6. Screw driver (10 a) for inserting the screw into the acetabular plate (P) holes and Spanner (9) for tightening of nuts (FIG. 10 c).

Method of Fixation of the Acetabular Plate—FIG. 9 and FIG. 10.

This involves holding of the plate and reduction of fracture using asymmetrical clamp (1) and clamp forceps (2) and resting of the acetabular plate (P) on the pelvic brim (FIG. 9a). This is followed by insertion of single cortical screw in the screw hole (SH1) at upper extended portion (A) and in anterior screw hole (SH2) for holding and supporting the acetabular plate (P) with bone. Alignment of the jig with compression sleeve and trocar (3) with acetabular plate (P) is carried out by resting the jig on the required hole (SH2) FIG. 9 b. For drilling of a hole from the outer cortex of bone, insertion of drill with drill bit (4) into the jig is carried out. Drilling is performed with help of guide wire (5). FIG. 9 c, d. Once drilling is over, bi-cortical screw (bolt) (6) with washer is inserted into the drilled hole and smaller screw with nut shaped head, is inserted from other end of bi-cortical guide wire (6). Then spanner is used for tightening of nut. FIG. 10 a. If further fixation is required, a single cortical screw can be inserted into the other screw holes of the acetabular plate from inner cortex of the bone for providing proper grip with bone.

A second embodiment of the present invention is shown in FIG. 11. It differs from the first embodiment (FIG. 7) in three aspects viz.
Upper portion is made more compact: In first embodiment (FIG. 7), the upper part A is broad, whereas in second embodiment (FIG. 11) the upper part A1 is ‘clipped’ and made more compact, without affecting functionality.
Additional small holes are present in plate for ball spike fixation (BS1 to BS5) FIG. 12
Presence of locking holes with internal screw threads (F1 and F4), FIG. 12.

The plate of the second embodiment (FIG. 11) is further described as below:
i. Flange (A1) contains four screw holes (F1, F2, F3 and F4). Holes F1 and F4 are locking holes with 4.5 mm and 3.5 mm diameter respectively that contain internal threads and allow the insertion of screws in predefined positions only. Holes (F2) and (F3) are normal 3.5 mm screw holes without any internal threads.
ii. Lower extended portion (B1) and (C1) contain two holes i.e. (LB1 and LB2) and (LC1 and LC2) respectively for inserting screws.
iii. The brim portion (D1) of the acetabular plate (P1) rests on the brim of the acetabular region in pelvic. The brim portion (D1) is anatomically shaped and brim specific. It contains multiple screw holes for providing proper hold of the plate with bone. Brim portion (D1) contains two dual cortical screw holes (DC1) and (DC2) into which cannulated screws are inserted into pelvic brim through these holes. Brim portion also contains five 3.5 mm screw holes (BR1, BR2, BR3, BR4 and BR5). This portion also contains five Small holes (BS1, BS2, BS3, BS4, BS5) that perform the function of temporary positioning of plate with kirschner wire or K-wire and to aid reduction by compression of the plate over the brim by using a ball spike (FIG. 12, E1).

Further, the thickness of acetabular plate is kept at 2 mm and made up of high strength medical grade stainless steel material that does not allow the plate to bend under the weight of patient.

Use of Uniquely Designed Cannulated Screw Set (Nut and Bolt) for Plate Fixation

In fixing of the acetabular plate of present invention, dual cortical cannulated screws are used having structure as shown in FIG. 13. The screw (bolt) is cannulated (hollow) with a small hole in the head to enable passing of guide wires. The screw set comprises a bigger screw called bolt (C_{BS}) and smaller screw called nut (C_{SS}). The bigger screw (bolt) (C_{BS}) contains a washer (W). The diameter of the cannula is such that the smaller screw can be fitted inside it. The smaller screw (nut) (C_{SS}) is without any washer and has a square head instead of a round head. Square shape head of small screw (C_{SS}) gives easy purchase (holding/compression) of screw when inserting into the bone over the guide wire. The smaller screw is screwed into the hollow cavity or cannula by internal threads (IT) of the bigger screw (C_{BS}), to maintain the required compression (tightening). Small screw fitted in big screw is shown in FIG. 13 (c).

In a third embodiment of the present invention (FIG. 14), the central brim portion (B2) of the plate is anatomically contoured to match shape of the pelvic brim to enable easy, accurate and quick fit of the plate. The brim portion also contains a screw line of several holes in which screws can be fitted and which is specifically created to facilitate the screw insertion from inside towards the outer cortex. It also has one big hole for trans-fixation screw and three small holes for 3.5 mm screws. Pubic arm or extended portion of the brim portion has 4-5 holes as per plate size. Upper flange (A2) of the body is having multiple screw hole line where the foremost posterior screw is to stabilize the plate for correct/optimum anatomical positioning of the plate. This has been achieved in a simple manner by giving threads in the hole, so that when screw is inserted, it is inserted easily and quickly at a particular position only.

In a fourth embodiment, the plate has plurality of holes on the upper flange which can vary as per size of the plate—small, medium or large. However, irrespective of the size of the plate, there are two holes on the flange of the plate which are locking holes—one along the upper rim (F1) and the second one below it (F4) of FIG. 12, which is positioned to typically lie on the anatomical brim.

Both holes have critical functionality, allowing correct and quick positioning the plate during surgery, thus drastically reducing surgery time, apart from enhancing accurate plate fixation in an easy and convenient manner for the surgeon.

The critical functionality of the two locking holes is further elaborated as below:
i. Locking hole one—present on the upper flange: It permits the ‘screwing into’ of a specially designed locking sleeve (hollow). The sleeve consists of two parts—an elongated holding rod with screw threaded terminal end which gets ‘screwed’ into the locking hole and a head which permits firm gripping. Once the sleeve is screwed into the plate vide the locking hole one, it allows the plate to be easily and comfortably held by the surgeon and allows insertion of the plate from the lateral approaches of the acetabulum in an easy and quick manner.
ii. Locking hole two—present below the first hole and positioned centrally on the plate so as to typically lie on the anatomical brim. It permits the ‘screwing into’ of the specially designed locking sleeve as described above. Once the sleeve is screwed into the plate vide the locking hole, it allows the plate to be easily and comfortably held by the surgeon and inserted from the medial approaches of the acetabulum in an easy and quick manner. This locking sleeve (locked on the brim by screwing), not only allows the plate to be positioned from the medial surgical approach but also allows the drill to be directed for insertion of the dual cortical screw described earlier (FIG. 13). Further, the distance between the two lower arms of the plate can be widened to offer the added and distinct advantage of easy fracture reduction. This is because when the in between space is increased it allows direct reduction of the fracture fragments.

The basic structure of all the plates is the same i.e. anatomical brim specificity and dual cortical fixation. However, in embodiments, there are minor improvements and modifications relating to additional holes for insertion of ball spikes to temporarily hold the plates while fixation is being carried out and also presence of additional holes with internal screw threads.

Regarding the method of fixation, it is common to all the plates and the embodiments. The cannulated screw set is also the same for all the plates.

Novelty

The novelty of the present invention lies in disclosing a novel acetabular compression plate with anatomical shape corresponding to pelvic brim and dual cortical fixation for reconstructing acetabular fractures in both osteoporotic bone and normal healthy bone. In the existing surgical methods for acetabular fractures only single cortex fixation or single cortex screws are used for fixing plates. In case of osteoporotic bone these screws lose their grip with bone and eventually with the plate also, which leads to loosening of the plate. As a result, these plates are required to be replaced. In case of the anatomical and brim specific novel acetabular plate of present invention, there is no need of replacement as plate does not become loose at all owing to proper and firm grip/bone compression, due to fixation from both sides—outer and inner (bi-conical fixation). Further, the fitting of the plate is quick and easy, resulting in drastic reduction of surgery time.

Inventive Step

The technical advancement of knowledge lies in disclosing an improved plate for reconstructing acetabular fractures in both osteoporotic bone and normal healthy bone, in a fast, efficient and accurate manner. The plate offers the technical advantage of dual cortical fixation due to which it does not become loose and hence does not require replacement after some time, which is a common problem in existing plates. This has been achieved by making the shape of the plate anatomical and brim specific, so that it completely rests on the brim of the pelvis, giving significantly enhanced strength to the fractured bone. The plate is held with bone using bi-cortical purchase/fixation that too from the outer cortex of the bone.

The Drilling Through Outer Cortex of the Bone is Done First Time by the Inventor for Providing a Bi-Cortical Purchase in the Acetabular Region of the Pelvis.

A bi-cortical screw is used with washer and is held in the brim of the pelvis which is very strong as compared to other bones of the acetabular region. This screw is tightened from both side of bone so as to provide proper compression and fixation of acetabular plate with the bone. Owing to simplicity of shape and ease of manufacturing, the invention drastically reduces time of surgery from 2.5-3 hours to less than one hour. The plate is simple, easy to manufacture and thus has economic value also.

### INDUSTRIAL APPLICATION

The acetabular plate is easy to manufacture at industrial level. It has widespread application in the treatment of acetabular/pelvic region fractures in normal as well as osteoporotic bones.

The above description of the invention along with embodiments is the best-contemplated mode of carrying out the invention and should not be construed in a limiting sense. The description is made for the purpose of illustrating the general principles of the invention relating to anatomical specificity of the plate design, which results in easy, quick and accurate fixation of the plate drastically reducing surgery time and secondly, relating to dual cortical fixation which enables strong grip even in osteoporotic bone, thus enabling repair of acetabular fractures even in patients with osteoporosis. The dimensions etc. can be varied by those skilled in the art without departing from the scope of the invention.

1. An acetabular compression plate with dual cortical fixation mechanism for the repair of acetabular fractures wherein the plate has a shape corresponding to entire surface of pelvic brim including top, inside and especially the edge which is always in line of fracture causing force and consists of five parts as below:
i. Upper extended portion (A) consisting of elongated flange which is longitudinally raised to fit over pelvic brim and possesses plurality of holes with screw threads at a pre-decided angle;
ii. Lower extended portion (B) consisting of short downward arm with plurality of holes with screw threads at a pre-decided angle;
iii. Lower extended portion (C) consisting of short downward arm with plurality of holes with screw threads at a pre-decided angle;
iv. Central Brim portion (D) having anatomical shape corresponding to the pelvic brim and possessing plurality of holes with screw threads at a pre-decided angle;
v. Notched part (N) which fits into pelvic brim in the posterior part of the brim, so that the plate fits anatomically over the brim and does not slide back, thus retaining its correct anatomical position and
wherein dual cortical fixation mechanism consists of:
Passing a special cannulated screw (FIG. 13) which fit into each other and allow passage of guide wire through them, through holes with screw threads at a pre-decided angle due to which the cannulated screw position gets locked at a particular angle;
Insertion of a nut inside the hollow screw after fixation of the plate, which ensures firm grip from both inside and outside, thus ensuring fracture stability.

2. An acetabular compression plate with dual cortical fixation mechanism for the repair of acetabular fractures wherein the plate has a shape corresponding to entire surface of pelvic brim including top, inside and especially the edge which is always in line of fracture causing force, as illustrated in FIG. 11 and consists of five parts as below:
i. Upper extended portion (A1) consisting of elongated flange with plurality of holes with screw threads at a pre-decided angle;
ii. Lower extended portion (B1) consisting of short downward arm with plurality of holes with screw threads at a pre-decided angle;
iii. Lower extended portion (C1) consisting of short downward arm with plurality of holes with screw threads at a pre-decided angle;
iv. Central Brim portion (D1) having anatomical shape corresponding to the pelvic brim and possessing plurality of holes with screw threads at a pre-decided angle;
v. Notched part (N) which fits into pelvic brim in the posterior part of the brim, so that the plate fits anatomically over the brim and does not slide back, thus retaining its correct anatomical position and
wherein dual cortical fixation mechanism consists of:
Passing a special cannulated screw (FIG. 13) which fit into each other and allow passage of guide wire through them, through holes with screw threads at a pre-decided angle due to which the cannulated screw position gets locked at a particular angle;
Insertion of a nut inside the hollow screw after fixation of the plate, which ensures firm grip from both inside and outside, thus ensuring fracture stability.

3. An acetabular compression plate with dual cortical fixation mechanism for the repair of acetabular fractures wherein the plate has a shape corresponding to entire surface of pelvic brim including top, inside and especially the edge which is always in line of fracture causing force, as illustrated in FIG. 14 and consists of five parts as below:
i. Upper extended portion (A2) consisting of elongated flange with plurality of holes with screw threads at a pre-decided angle;
ii. Lower extended portion (B2) consisting of short downward arm with plurality of holes with screw threads at a predecided angle;
iii. Lower extended portion (C2) consisting of short downward arm with plurality of holes with screw threads at a predecided angle;
iv. Central Brim portion (D2) having anatomical shape corresponding to the pelvic brim and possessing plurality of holes with screw threads at a pre-decided angle;
v. Notched part (N) which fits into pelvic brim in the posterior part of the brim, so that the plate fits anatomically over the brim and does not slide back, thus retaining its correct anatomical position and
wherein dual cortical fixation mechanism consists of:
Passing a special cannulated screw (FIG. 13) which fit into each other and allow passage of guide wire through them, through holes with screw threads at a pre-decided angle due to which the cannulated screw position gets locked at a particular angle;
Insertion of a nut inside the hollow screw after fixation of the plate, which ensures firm grip from both inside and outside, thus ensuring fracture stability.

4. The plate as claimed in claim 1, wherein out of the plurality of holes present in the upper extended portion, two holes viz. F1 and F4 as illustrated in FIG. 12 are locking holes with screw threads.

5. The plate as claimed in claim 2, wherein out of the plurality of holes present in the upper extended portion, two holes viz. F1 and F4 as illustrated in FIG. 12 are locking holes with screw threads.

6. The plate as claimed in claim 3, wherein out of the plurality of holes present in the upper extended portion, two holes viz. F1 and F4 as illustrated in FIG. 12 are locking holes with screw threads.
